(19) United States
(12) Patent Application Publication
(10) Pub. No.: US 2023/0201529 A1
(43) Pub. Date: Jun. 29, 2023

(54) CATHETER, CATHETER ASSEMBLY, AND CATHETER SYSTEM

(71) Applicant: TERUMO KABUSHIKI KAISHA, Tokyo (JP)

(72) Inventors: Shota YAMAMOTO, Mitaka-shi (JP); Takayuki YOKOTA, Chuo-shi (JP)

(73) Assignee: TERUMO KABUSHIKI KAISHA, Tokyo (JP)

(21) Appl. No.: 18/111,482

(22) Filed: Feb. 17, 2023

Related U.S. Application Data
(63) Continuation of application No. PCT/JP2021/030095, filed on Aug. 18, 2021.

(30) Foreign Application Priority Data
Aug. 24, 2020 (JP) 2020-140575

(51) Int. Cl. A61M 25/00; A61M 25/06

(52) U.S. Cl. CPC A61M 25/0074; A61M 25/0612; A61M 2025/0008

(57) ABSTRACT

A catheter includes: a shaft to be inserted into a blood vessel, the shaft being flexible and having a tubular shape. The shaft comprises: a light emitting portion that is located at a distal end portion of the shaft and is configured to emit near-infrared light by being irradiated with light, and a non-light emitting portion that extends from the light emitting portion toward a proximal end side of the shaft and does not emit the near-infrared light. A length of the light emitting portion in an extending direction of the shaft is six times or less an outer diameter of the non-light emitting portion in an extending direction of the shaft.

### CROSS-REFERENCE TO RELATED APPLICATIONS

This application is a bypass continuation of PCT Application No. PCT/JP2021/030095, filed on Aug. 18, 2021, which claims priority to Japanese Application No. JP2020-140575, filed on Aug. 24, 2020. The contents of these applications are hereby incorporated by reference in their entirety.

### BACKGROUND

The present disclosure relates to a catheter, a catheter assembly, and a catheter system.

In recent years, a blood vessel visualization system that visualizes a blood vessel with an image (received light image) obtained by irradiating a living body site with near-infrared light and receiving the near-infrared light guided through the living body site has been developed. The blood vessel visualization system is used, for example, when a shaft of a catheter is inserted into a blood vessel (see JP 2014-136115 A). The shaft of the catheter of JP 2014-136115 A includes a light emitting portion that emits near-infrared light. When such a catheter is used, because near-infrared light is absorbed by blood, a site of the light emitting portion located outside a blood vessel is displayed to be relatively bright, and a site of the light emitting portion inserted into the blood vessel is displayed to be relatively dark in a received light image.

### SUMMARY

In the above-described conventional technique, the light emitting portion is disposed over a relatively long range from a distal end to a proximal end side of the shaft. In such a catheter, when a distal end portion of the shaft (a distal end portion of the light emitting portion) is inserted into a blood vessel, a proximal end side of the light emitting portion is located outside the blood vessel. In this case, only the distal end portion of the shaft is darkened in a received light image. Therefore, it is difficult for a user to find that the distal end portion of the shaft has been inserted into a blood vessel (blood vessel securement of the catheter) at a relatively early stage by viewing the received light image.

Embodiments of the present disclosure have been developed in view of such a problem, and an object of certain embodiments is to provide a catheter, a catheter assembly, and a catheter system that can allow a user to easily determine blood vessel securement of the catheter at a relatively early stage by viewing the received light image.

According to a first aspect of the present disclosure, a catheter includes a shaft to be inserted into a blood vessel, the shaft being flexible and formed in a tubular shape, in which the shaft includes: a light emitting portion that is located at a distal end of the shaft and emits near-infrared light by being irradiated with light; and a non-light emitting portion that extends from the light emitting portion toward a proximal end side of the shaft and does not emit the near-infrared light, and the light emitting portion has a length of 7 mm or less or six times or less the outer diameter of the non-light emitting portion in an extending direction of the shaft.

According to a second aspect of the present disclosure, a catheter assembly includes the above-described catheter and a needle body inserted into the catheter.

According to a third aspect of the present disclosure, a catheter system includes: the above-described catheter; an irradiation unit that irradiates a living body site including a blood vessel into which the catheter is inserted with light; a light receiving unit that receives the light guided from the living body site and the near-infrared light emitted by the light emitting portion; and an image display unit that displays a received light image created on the basis of the light and the near-infrared light received by the light receiving unit.

According to certain embodiment of the present disclosure, because the length of the light emitting portion in the extending direction of the shaft is 7 mm or less or six times or less the outer diameter of the non-light emitting portion, the entire light emitting portion can be located in a blood vessel when a distal end portion of the shaft is inserted into the blood vessel. In this case, the entire light emitting portion is darkened in a received light image. Therefore, a user can easily find blood vessel securement of the catheter at a relatively early stage by viewing the received light image.

### BRIEF DESCRIPTION OF DRAWINGS

FIG. 1 is a schematic configuration diagram of a catheter system according to an embodiment of the present invention;

FIG. 2 is an exploded perspective view of the catheter assembly of FIG. 1;

FIG. 3 is a longitudinal cross-sectional view of a distal end portion of the catheter assembly of FIG. 1;

FIG. 4 is a first explanatory diagram of a procedure for the catheter assembly of FIG. 1 to puncture a blood vessel;

FIG. 5 is a received light image in the state of FIG. 4;

FIG. 6 is a second explanatory diagram of the procedure for the catheter assembly of FIG. 1 to puncture the blood vessel;

FIG. 7 is a received light image in the state of FIG. 6;

FIG. 8A is a longitudinal cross-sectional view of a distal end portion of a catheter assembly including a shaft including a light emitting portion according to a first modification; and

FIG. 8B is a longitudinal cross-sectional view of a distal end portion of a catheter assembly including a shaft including a light emitting portion according to a second modification.

### DETAILED DESCRIPTION

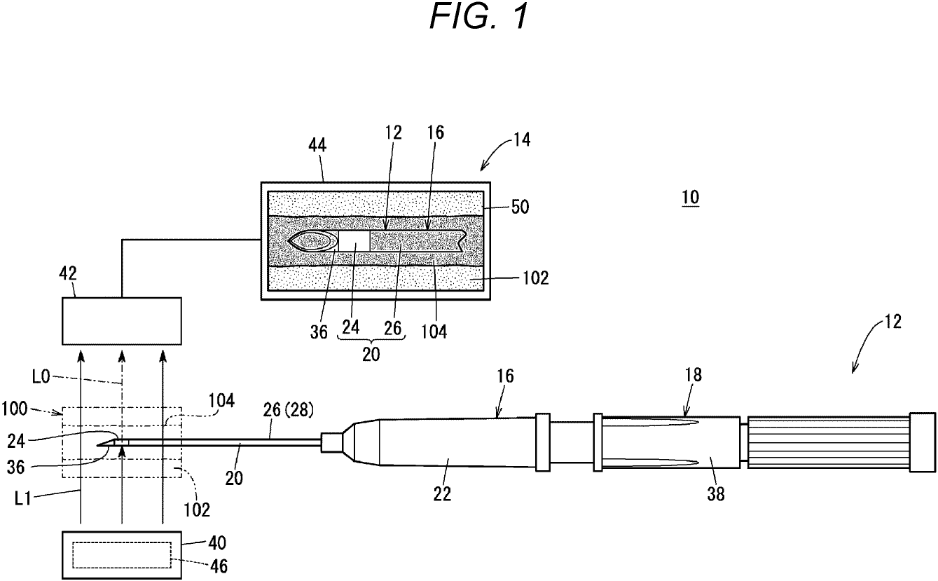

As illustrated in FIG. 1, a catheter system 10 according to an embodiment of the present invention includes: a catheter assembly 12 capable of puncturing a blood vessel 104 of a living body site 100; and a blood vessel visualization system 14 for visualizing the blood vessel 104.

The catheter assembly 12 is formed as an indwelling needle for administering an infusion (drug) into the blood vessel 104 of the living body site 100. Note that the catheter assembly 12 is not limited to one that administers a drug.

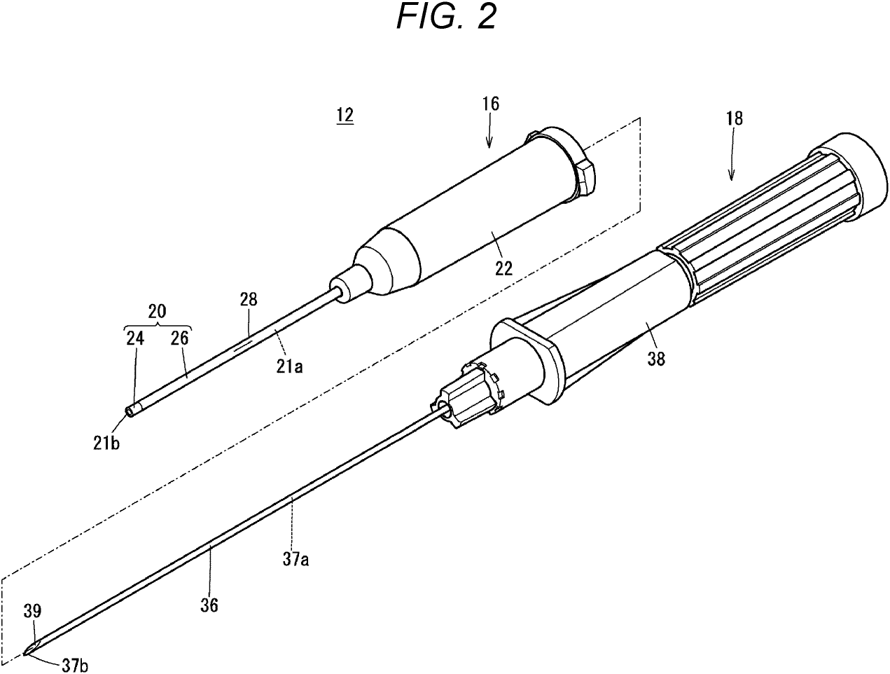

As illustrated in FIGS. 1 and 2, the catheter assembly 12 includes a catheter 16 and a puncture needle 18. The catheter 16 includes a shaft 20 to be inserted into the blood vessel 104 and a catheter hub 22 disposed at a proximal end portion of the shaft 20.

The shaft 20 is flexible. The shaft 20 is a tubular member that can be continuously inserted into the blood vessel 104 (for example, basilic vein, median cubital vein, or cephalic vein) of the living body site 100. The shaft 20 has a lumen 21a extending in an axial direction over the entire length thereof (see FIG. 2).

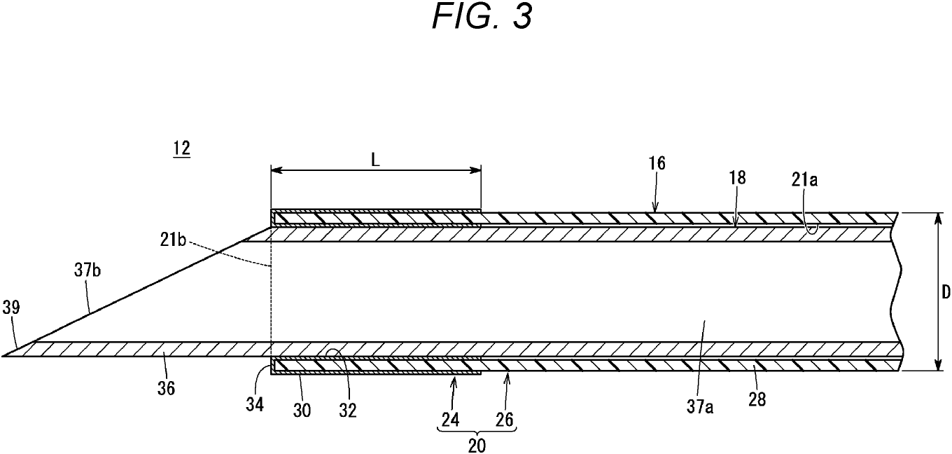

In FIG. 3, a distal end opening 21b communicating with the lumen 21a is formed at a distal end of the shaft 20. The shaft 20 includes: a light emitting portion 24 that is located at a distal end of the shaft 20 and emits near-infrared light L0 (see FIG. 1) by being irradiated with light; and a non-light emitting portion 26 that extends from the light emitting portion 24 toward a proximal end side of the shaft 20 and does not emit near-infrared light L0. In addition, the shaft 20 includes a shaft body 28 extending from a distal end portion to a proximal end of the shaft 20. The shaft body 28 forms the non-light emitting portion 26.

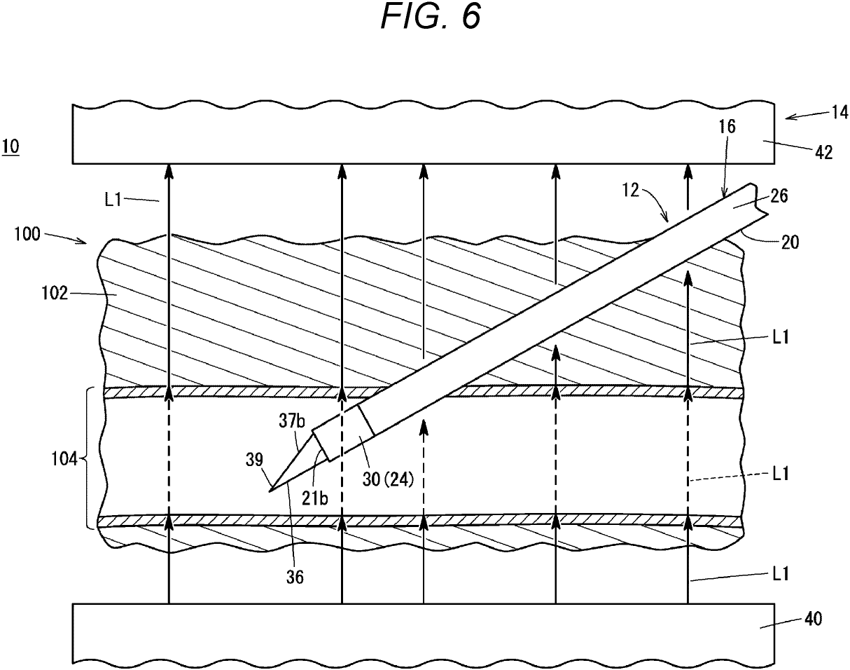

The light emitting portion 24 extends from a distal end of the shaft 20 toward a proximal end thereof. A length L of the light emitting portion 24 in the extending direction of the shaft 20 is set to 7 mm or less. As a result, when a distal end portion of the catheter assembly 12 punctures the blood vessel 104, the entire light emitting portion 24 can be located in the blood vessel 104 (see FIG. 6). The length L of the light emitting portion 24 is preferably set to 5 mm or less, and more preferably set to 3 mm or less. In this case, when the distal end portion of the catheter assembly 12 punctures the blood vessel 104, the entire light emitting portion 24 is easily located in the blood vessel 104. Note that the length L of the light emitting portion 24 is preferably set to 1 mm or more in consideration of visibility of the light emitting portion 24 in an image (received light image 50) obtained by the blood vessel visualization system 14 illustrated in FIG. 1.

In addition, the length L of the light emitting portion 24 may be set to six times or less the outer diameter D of the non-light emitting portion 26 (shaft body 28). As a result, when a distal end portion of the catheter assembly 12 punctures the blood vessel 104, the entire light emitting portion 24 can be located in the blood vessel 104 (see FIG. 6). The length L of the light emitting portion 24 is preferably set to four times or less the outer diameter D of the non-light emitting portion 26, and more preferably set to two times or less the outer diameter D of the non-light emitting portion 26. In this case, when the distal end portion of the catheter assembly 12 punctures the blood vessel 104, the entire light emitting portion 24 is easily located in the blood vessel 104. Note that the length L of the light emitting portion 24 is preferably set to one time or more the outer diameter D of the non-light emitting portion 26 in consideration of visibility of the light emitting portion 24 in the image (received light image 50) obtained by the blood vessel visualization system 14 illustrated in FIG. 1.

The length L of the light emitting portion 24 is appropriately set depending on the outer diameter of the blood vessel 104 into which the shaft 20 is inserted, the thickness of the shaft 20, a puncture angle, and the like.

In FIG. 1, a peak wavelength of near-infrared light L0 emitted by the light emitting portion 24 is within a range of wavelengths whose light is easily absorbed by hemoglobin in blood and easily passes through a living body tissue 102 (skin tissue, muscle tissue, and the like). In general, the living body tissue 102 easily transmits light having a wavelength of 700 nm or more and 1000 nm or less. In addition, reduced hemoglobin of blood flowing in the vein easily absorbs light having a wavelength around 660 nm. Furthermore, oxidized hemoglobin of blood flowing in the artery easily absorbs light having a wavelength around 940 nm.

Therefore, the peak wavelength of near-infrared light L0 emitted by the light emitting portion 24 is preferably within a range of 700 nm or more and 1000 nm or less. In a case of the catheter assembly 12 used for puncturing the vein (catheter to be inserted into the vein), the peak wavelength of near-infrared light L0 emitted by the light emitting portion 24 is preferably within a range of 700 nm or more and 800 nm or less, and more preferably within a range of 700 nm or more and 750 nm or less. In a case of the catheter assembly 12 used for puncturing the artery (catheter to be inserted into the artery), the peak wavelength of near-infrared light L0 emitted by the light emitting portion 24 is preferably within a range of 800 nm or more and 1000 nm or less, more preferably within a range of 850 nm or more and 950 nm or less, and still more preferably around 940 nm.

As illustrated in FIG. 3, the light emitting portion 24 includes: an outer light emitting layer 30 formed by applying a light emitting material to an outer surface of the distal end portion of the shaft body 28; an inner light emitting layer 32 formed by applying a light emitting material to an inner surface of the distal end portion of the shaft body 28; and a distal end light emitting portion 34 formed by applying a light emitting material to a distal end surface of the shaft body 28. Such a light emitting portion 24 can be easily obtained by immersing the distal end portion of the shaft body 28 in a liquid light emitting material.

Each of the outer light emitting layer 30 and the inner light emitting layer 32 annularly extends in a circumferential direction of the shaft body 28. Note that each of the outer light emitting layer 30 and the inner light emitting layer 32 may extend in a length of less than 360° in the circumferential direction of the shaft body 28 (for example, in a semicircular shape). In addition, each of the outer light emitting layers 30 and inner light emitting layers 32 may be provided in a plurality of locations with a gap between each other in the circumferential direction of the shaft body 28.

The light emitting portion 24 does not have to include both the outer light emitting layer 30 and the inner light emitting layer 32. In addition, the light emitting portion 24 does not have to include the distal end light emitting portion 34. That is, the light emitting portion 24 only needs to include at least one of the outer light emitting layer 30 and the inner light emitting layer 32.

In FIG. 1, as the light emitting material, a fluorescent material (near-infrared fluorescent dye) that emits near-infrared light L0 by being irradiated with light L1 (for example, near infrared light or visible light) emitted by an irradiation unit 40 of the blood vessel visualization system 14 is used. Examples of such a fluorescent material include those described in JP 2014-136115 A.

As the light emitting material, a phosphorescent material that emits near-infrared light L0 by being irradiated with light L1 or light other than light L1 may be used. In a case of using such a phosphorescent material, by irradiating the light emitting portion 24 with light in advance (before performing a procedure of blood vessel puncture using the catheter system 10), the light emitting portion 24 can be made to emit light. As a result, near-infrared light can be effectively emitted from the light emitting portion 24 in the living body site 100. Examples of the phosphorescent material include that described by the following chemical formula.

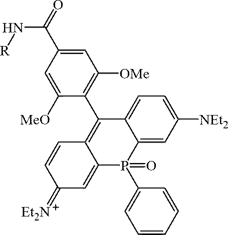

In FIG. 3, the shaft body 28 includes a flexible resin material. Examples of such a resin material include a fluorine-based resin such as polytetrafluoroethylene (PTFE), an ethylene-tetrafluoroethylene copolymer (ETFE), or a perfluoroalkoxy fluorine resin (PFA), an olefin-based resin such as polyethylene or polypropylene or a mixture thereof, polyurethane, polyester, polyamide, a polyether nylon resin, and a mixture of an olefin-based resin and an ethylene-vinyl acetate copolymer.

At least a portion forming the non-light emitting portion 26 in the shaft body 28 includes a material that absorbs light L1 emitted by the irradiation unit 40 of the blood vessel visualization system 14. Specific examples of the material (near-infrared light absorbing dye) that absorbs light L1 (for example, near-infrared light) include a cyanine dye, a phthalocyanine dye, a naphthalocyanine compound, a nickel dithiolene complex, a squarylium dye, a quinone-based compound, a diimmonium compound, and an azo compound. Such a near-infrared light absorbing dye is mixed (kneaded) with the resin material forming the shaft body 28. That is, the shaft body 28 (non-light emitting portion 26) contains the near-infrared light absorbing dye. Note that the near-infrared light absorbing dye may be applied to an outer surface of the shaft body 28 on a proximal end side of the light emitting portion 24.

As illustrated in FIG. 2, the catheter hub 22 is formed in a hollow shape (cylindrical shape). The catheter hub 22 is preferably made of a material harder than the shaft 20. A constituent material of the catheter hub 22 is not particularly limited, but for example, a thermoplastic resin such as polypropylene, polycarbonate, polyamide, polysulfone, polyarylate, a methacrylate-butylene-styrene copolymer, polyurethane, an acrylic resin, or an ABS resin can be suitably used.

The puncture needle 18 includes a needle body 36 and a needle hub 38 disposed at a proximal end portion of the needle body 36. The needle body 36 is a tubular member having rigidity capable of puncturing the living body site 100 (see FIG. 1). The needle body 36 has a lumen 37a extending in an axial direction. Examples of a material constituting the needle body 36 include a metal material such as stainless steel, aluminum, an aluminum alloy, titanium, or a titanium alloy. The needle body 36 is formed sufficiently longer than the shaft 20 and protrudes from the distal end opening 21b of the shaft 20 in an initial state (assembled state) of the catheter assembly 12 (see FIGS. 1 and 3).

In FIGS. 2 and 3, a blade surface 39 inclined with respect to an axis of the needle body 36 is formed at a distal end portion of the needle body 36. A distal end opening 37b communicating with the lumen 37a of the needle body 36 is formed on the blade surface 39.

In FIGS. 1 and 2, the needle hub 38 is formed in a hollow shape (tubular shape). A constituent material of the needle hub 38 may be the same as the constituent material of the above-described catheter hub 22. A proximal end portion of the needle body 36 is fixed to a distal end portion of the needle hub 38. The needle hub 38 functions as an operation portion of the catheter assembly 12.

As illustrated in FIG. 1, the blood vessel visualization system 14 includes the irradiation unit 40, a light receiving unit 42, and an image display unit 44. The irradiation unit 40 irradiates the living body site 100 (visualization target) punctured by the catheter assembly 12 with light L1. The irradiation unit 40 includes a light source 46 that emits light L1. The light source 46 emits near-infrared light as light L1. For example, the near-infrared light emitted by the light source 46 preferably has a wavelength within a range of 700 nm or more and 2500 nm or less, more preferably has a wavelength within a range of 700 nm or more and 1400 nm or less, and still more preferably has a wavelength within a range of 780 nm or more and 1050 nm or less. Such near-infrared light is absorbed by hemoglobin of blood. The light source 46 may emit visible light (not including near-infrared light). In addition, the light source 46 may emit light including both near-infrared light and visible light.

The light receiving unit 42 is disposed on a side opposite to the irradiation unit 40 with the living body site 100 interposed therebetween. In other words, the irradiation unit 40 and the light receiving unit 42 are disposed so as to face each other with the living body site 100 interposed therebetween. The light receiving unit 42 is a camera (imaging unit) that receives light L1 guided from the living body site 100 and near-infrared light L0 emitted by the light emitting portion 24. As the light receiving unit 42, for example, a CCD camera for near-infrared light is used. The image display unit 44 displays an image (received light image 50) created on the basis of light L1 and near-infrared light L0 received by the light receiving unit 42. The image display unit 44 may be a goggle that can be attached to and detached from a person or a stationary display.

Next, a procedure of blood vessel puncture using the catheter system 10 will be described.

As illustrated in FIG. 3, in an initial state of the catheter assembly 12, the blade surface 39 protrudes in a distal end direction from the distal end opening 21b of the shaft 20 in a state of facing upward.

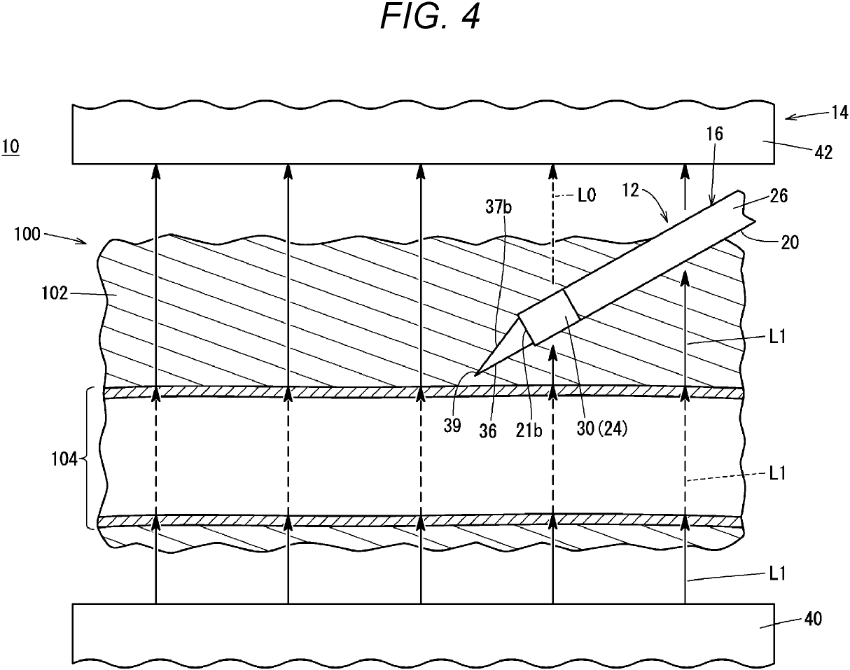

First, a user sets the blood vessel visualization system 14. Specifically, as illustrated in FIG. 4, the irradiation unit 40 is disposed below the living body site 100 (for example, a forearm of a human body) to be punctured, and the light receiving unit 42 is disposed above the living body site 100. Then, the irradiation unit 40 irradiates the living body site 100 with light L1, and a distal end portion of the catheter assembly 12 punctures the living body site 100.

Then, light L1 emitted by the irradiation unit 40 passes through the living body tissue 102 (for example, a skin tissue or a muscle tissue) of the living body site 100 while being scattered, and light L1 (transmission light) that has passed through the living body tissue 102 is received by the light receiving unit 42. At this time, hemoglobin of blood flowing in the blood vessel 104 absorbs light L1. In addition, the light emitting portion 24 of the catheter 16 emits near-infrared light L0 by being irradiated with light L1. Near-infrared light L0 emitted by the light emitting portion 24 is received by the light receiving unit 42. Furthermore, the non-light emitting portion 26 of the catheter 16 absorbs light L1, and the needle body 36 reflects (does not transmit) light L1.

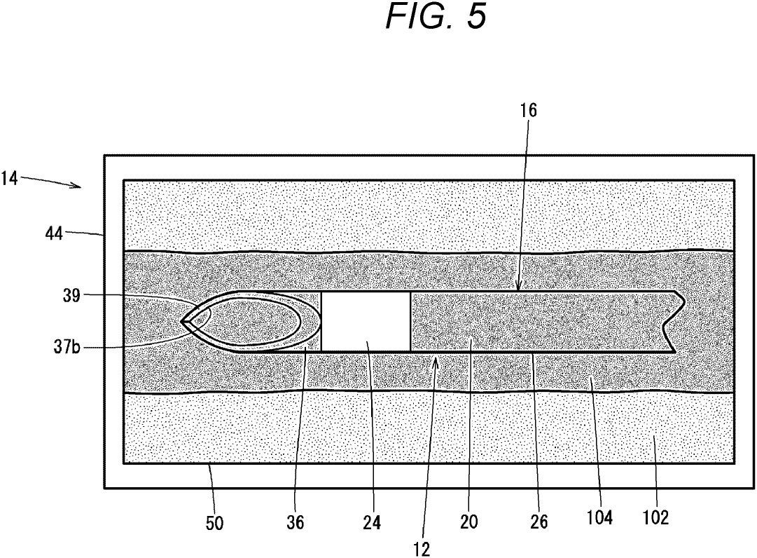

As a result, as illustrated in FIG. 5, the received light image 50 created on the basis of light L1 and near-infrared light L0 received by the light receiving unit 42 is displayed on the image display unit 44. In the received light image 50, the living body tissue 102 (other than the blood vessel 104), the blood vessel 104, the light emitting portion 24, the non-light emitting portion 26, and the needle body 36 are displayed. Specifically, in the received light image 50, the light emitting portion 24 is displayed brightest (white), and the living body tissue 102 (other than the blood vessel 104) is displayed darker than the light emitting portion 24. In addition, in the received light image 50, the blood vessel 104, the non-light emitting portion 26, and the needle body 36 are displayed darker than the living body tissue 102. Note that the blood vessel 104, the non-light emitting portion 26, and the needle body 36 have substantially the same brightness (luminance). As a result, a user can easily and clearly distinguish between the blood vessel 104 and the light emitting portion 24 in the received light image 50.

Subsequently, as illustrated in FIG. 6, when the distal end portion of the catheter assembly 12 is inserted into the blood vessel 104, blood in the blood vessel 104 flows into the lumen 37a (see FIG. 3) of the needle body 36 from the distal end opening 37b of the needle body 36. At this time, because the entire light emitting portion 24 is located in the blood vessel 104, light L1 emitted by the light source 46 is not guided to the light emitting portion 24. Therefore, in a case in which the light emitting portion 24 is made of a fluorescent material, near-infrared light L0 is not emitted from the light emitting portion 24. On the other hand, in a case in which the light emitting portion 24 is made of a phosphorescent material, near-infrared light L0 emitted from the light emitting portion 24 is absorbed by hemoglobin of blood, and therefor does not reach the light receiving unit 42.

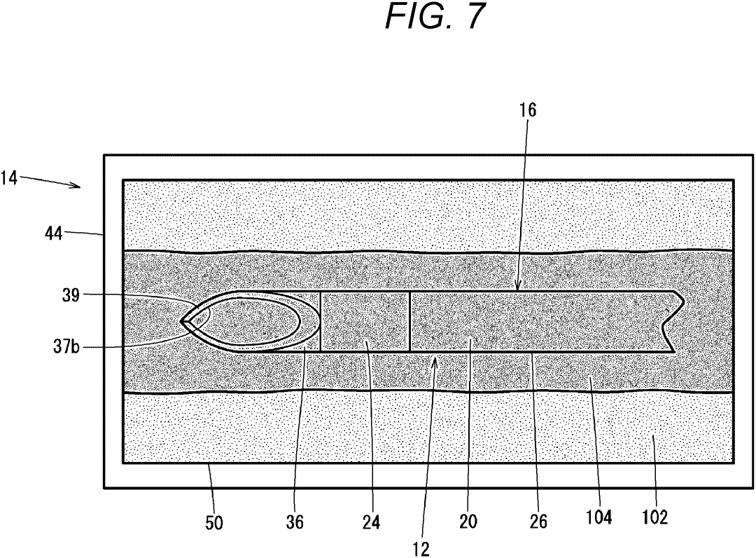

Therefore, as illustrated in FIG. 7, an appearance of the light emitting portion 24 changes (the light emitting portion 24 is darkened) in the received light image 50. In other words, the light emitting portion 24 and the blood vessel 104 have substantially the same brightness. Therefore, a user can easily find that a distal end portion of the catheter assembly 12 (a distal end portion of the catheter 16 and a distal end portion of the needle body 36) has been inserted into the blood vessel 104 at a relatively early stage by viewing the received light image 50. In other words, the user can easily find blood vessel securement of the catheter assembly 12 (blood vessel securement of the catheter 16 and blood vessel securement of the needle body 36) at a relatively early stage by viewing the received light image 50.

Thereafter, the user removes the puncture needle 18 in a state where the distal end portion of the shaft 20 is indwelled in the blood vessel 104, and administers a drug into the blood vessel 104 via the catheter 16.

The catheter 16, the catheter assembly 12, and the catheter system 10 according to the present embodiment have the following effects.

The shaft 20 includes: the light emitting portion 24 that is located at a distal end of the shaft 20 and emits near-infrared light L0 by being irradiated with light (for example, light L1); and the non-light emitting portion 26 that extends from the light emitting portion 24 toward a proximal end side of the shaft 20 and does not emit near-infrared light L0. The length L of the light emitting portion 24 in an extending direction of the shaft 20 is 7 mm or less or six times or less the outer diameter D of the non-light emitting portion 26.

According to such a configuration, when the distal end portion of the shaft 20 is inserted into the blood vessel 104, the entire light emitting portion 24 can be located in the blood vessel 104. In this case, the entire light emitting portion 24 is darkened in the received light image 50. Therefore, a user can easily find blood vessel securement of the catheter 16 at a relatively early stage by viewing the received light image 50.

The non-light emitting portion 26 is disposed over the entire range from a proximal end of the light emitting portion 24 to a proximal end of the shaft 20.

According to such a configuration, in a state before the light emitting portion 24 is inserted into the blood vessel 104, a user can easily distinguish between the light emitting portion 24 and the non-light emitting portion 26 in the received light image 50.

The shaft 20 includes the shaft body 28 forming the non-light emitting portion 26. The light emitting portion 24 includes a light emitting layer (at least one of the inner light emitting layer 32 and the outer light emitting layer 30) formed by applying a light emitting material to at least one of an inner surface and an outer surface of a distal end portion of the shaft body 28.

According to such a configuration, the light emitting portion 24 can be easily formed. In addition, in a case in which the light emitting portion 24 includes the outer light emitting layer 30, near-infrared light L0 emitted by the outer light emitting layer 30 can be efficiently guided to the light receiving unit 42. Furthermore, in a case in which the light emitting portion 24 includes the inner light emitting layer 32, contact of the light emitting material with the living body site 100 can be suppressed.

The light emitting material includes a fluorescent material or a phosphorescent material. In this case, the light emitting portion 24 that emits near-infrared light L0 can be easily obtained.

The non-light emitting portion 26 includes a material that absorbs light L1.

According to such a configuration, in a state before the light emitting portion 24 is inserted into the blood vessel 104, a user can more easily distinguish between the light emitting portion 24 and the non-light emitting portion 26 in the received light image 50.

A peak wavelength of near-infrared light L0 emitted by the light emitting portion 24 is within a range of 700 nm or more and 1000 nm or less.

According to such a configuration, near-infrared light L0 emitted by the light emitting portion 24 can easily pass through the living body tissue 102 (other than the blood vessel 104) and can be easily absorbed by hemoglobin of blood.

The catheter assembly 12 includes the catheter 16 and the needle body 36 inserted into the catheter 16.

According to such a configuration, a user can easily find that a distal end portion of the catheter assembly 12 (a distal end portion of the needle body 36 and a distal end portion of the shaft 20) has been inserted into the blood vessel 104 (blood vessel securement of the catheter assembly 12) at a relatively early stage by viewing the received light image 50.

The catheter system 10 includes: the catheter 16; the irradiation unit 40 that irradiates the living body site 100 including the blood vessel 104 into which the catheter 16 is inserted with light L1; the light receiving unit 42 that receives light L1 guided from the living body site 100 and near-infrared light L0 emitted by the light emitting portion 24; and the image display unit 44 that displays the received light image 50 created on the basis of light L1 and near-infrared light L0 received by the light receiving unit 42. The irradiation unit 40 and the light receiving unit 42 are disposed so as to face each other with the living body site 100 interposed therebetween.

According to such a configuration, the received light image 50 can be obtained by light L1 that has passed through the living body site 100. In this case, because reflected light reflected by a surface of the living body site 100 is not guided to the light receiving unit 42, the light emitting portion 24 can be clearly displayed in the received light image 50.

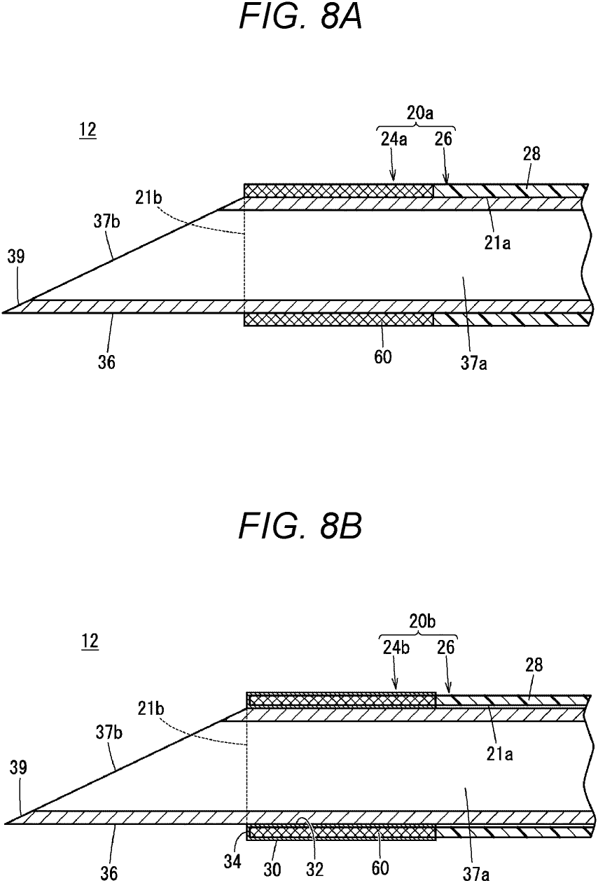

The catheter 16 may include a shaft 20a including a light emitting portion 24a illustrated in FIG. 8A. As illustrated in FIG. 8A, the light emitting portion 24a includes a light emitting wall portion 60 formed by inclusion of a light emitting material in a distal end portion of the shaft body 28. The light emitting wall portion 60 is formed by mixing (kneading) a light emitting material with a resin material forming the shaft body 28. As the light emitting material, the above-described fluorescent material or phosphorescent material can be used.

According to such a light emitting portion 24a, an effect similar to that of the above-described light emitting portion 24 is obtained.

The catheter 16 may include a shaft 20b including a light emitting portion 24b illustrated in FIG. 8B. As illustrated in FIG. 8B, the light emitting portion 24b includes the light emitting wall portion 60, the outer light emitting layer 30, the inner light emitting layer 32, and the distal end light emitting portion 34. According to such a light emitting portion 24b, an effect similar to that of the above-described light emitting portion 24 is obtained.

The present invention is not limited to the above-described embodiment, and various modifications can be made without departing from the gist of the present invention. The light receiving unit 42 may be a spectrum camera that disperses light for each wavelength and captures an image. In this case, the light receiving unit 42 can also cause the image display unit 44 to display an image by changing a color (or changing a color density) for each wavelength of received light. As a result, the living body tissue 102 (other than the blood vessel 104), the blood vessel 104, the light emitting portion 24, the non-light emitting portion 26, and the needle body 36 are displayed in different colors (or color densities) on the image display unit 44. The blood vessel visualization system 14 is not limited to a transmissive system that receives transmission light that has passed through the living body site 100 and displays the received light image 50, and may be a reflective system that receives reflected light reflected by the living body site 100 and displays the received light image 50.

The above embodiment is summarized as follows.

The above embodiment discloses a catheter (16) including a shaft (20, 20a, 20b) to be inserted into a blood vessel (104), the shaft being flexible and formed in a tubular shape, in which the shaft includes: a light emitting portion (24, 24a, 24b) that is located at a distal end portion of the shaft and emits near-infrared light (L0) by being irradiated with light (L1); and a non-light emitting portion (26) that extends from the light emitting portion toward a proximal end side of the shaft and does not emit the near-infrared light, and the light emitting portion has a length (L) of 7 mm or less or six times or less the outer diameter (D) of the non-light emitting portion in an extending direction of the shaft.

In the above-described catheter, the non-light emitting portion may be disposed over the entire range from a proximal end of the light emitting portion to a proximal end of the shaft.

In the above-described catheter, the shaft may include a shaft body (28) forming the non-light emitting portion, and the light emitting portion may include a light emitting layer (30, 32) formed by applying a light emitting material to at least one of an inner surface and an outer surface of a distal end portion of the shaft body.

In the above-described catheter, the shaft may include a shaft body forming the non-light emitting portion, and the light emitting portion (24a, 24b) may include a light emitting wall portion (60) formed by inclusion of a light emitting material in a distal end portion of the shaft body.

In the above-described catheter, the light emitting portion (24b) may include a light emitting wall portion formed by inclusion of a light emitting material in a distal end portion of the shaft body.

In the above-described catheter, the light emitting material may include a fluorescent material.

In the above-described catheter, the light emitting material may include a phosphorescent material.

In the above-described catheter, the non-light emitting portion may include a material that absorbs light.

In the above-described catheter, a peak wavelength of the near-infrared light emitted by the light emitting portion may be within a range of 700 nm or more and 1000 nm or less.

The above embodiment discloses a catheter assembly (12) including the above-described catheter and a needle body (36) inserted into the catheter.

The above embodiment discloses a catheter system (10) including: the above-described catheter; an irradiation unit (40) that irradiates a living body site (100) including a blood vessel into which the catheter is inserted with light (L1); a light receiving unit (42) that receives the light guided from the living body site and the near-infrared light emitted by the light emitting portion; and an image display unit (44) that displays a received light image (50) created on the basis of the light and the near-infrared light received by the light receiving unit.

The above-described catheter system may include a catheter assembly including the catheter and a needle body inserted into the catheter.

In the above-described catheter system, the irradiation unit and the light receiving unit may be disposed so as to face each other with the living body site interposed therebetween.

What is claimed is:

1. A catheter comprising:
a shaft to be inserted into a blood vessel, the shaft being flexible and having a tubular shape; wherein:
the shaft comprises:
a light emitting portion that is located at a distal end portion of the shaft and is configured to emit near-infrared light by being irradiated with light, and
a non-light emitting portion that extends from the light emitting portion toward a proximal end side of the shaft and does not emit the near-infrared light; and
a length of the light emitting portion in an extending direction of the shaft is 7 mm or less.

2. The catheter according to claim 1, wherein:
the non-light emitting portion is disposed over an entire length from a proximal end of the light emitting portion to a proximal end of the shaft.

3. The catheter according to claim 1, wherein:
the shaft comprises a shaft body;
the non-light emitting portion is formed by a portion of the shaft body; and
the light emitting portion comprises a light emitting layer on at least one of an inner surface or an outer surface of a distal end portion of the shaft body.

4. The catheter according to claim 3, wherein:
the light emitting portion further comprises a light emitting wall portion formed by inclusion of a light emitting material in a distal end portion of the shaft body.

5. The catheter according to claim 1, wherein:
the shaft comprises a shaft body;
the non-light emitting portion is formed by a portion of the shaft body, and
the light emitting portion comprises a light emitting wall portion formed by inclusion of a light emitting material in a distal end portion of the shaft body.

6. The catheter according to claim 3, wherein:
the light emitting material includes a fluorescent material.

7. The catheter according to claim 5, wherein:
the light emitting material includes a fluorescent material.

8. The catheter according to claim 3, wherein:
the light emitting material includes a phosphorescent material.

9. The catheter according to claim 5, wherein:
the light emitting material includes a phosphorescent material.

10. The catheter according to claim 1, wherein:
the non-light emitting portion includes a material that absorbs light.

11. The catheter according to claim 1, wherein:
a peak wavelength of the near-infrared light emitted by the light emitting portion is within a range of 700 nm or more and 1000 nm or less.

12. A catheter assembly comprising:
the catheter according to claim 1; and
a needle body inserted into the catheter.

13. A catheter system comprising:
the catheter according to claim 1;
an irradiation unit configured to irradiate a living body site including a blood vessel into which the catheter is inserted with light;
a light receiving unit configured to receive the light guided from the living body site and the near-infrared light emitted by the light emitting portion; and
an image display unit configured to display a received light image created on a basis of the light and the near-infrared light received by the light receiving unit.

14. The catheter system according to claim 14, comprising:
a catheter assembly comprising:
the catheter, and
a needle body inserted into the catheter.

15. The catheter system according to claim 14, wherein:
the irradiation unit and the light receiving unit are disposed so as to face each other with the living body site interposed therebetween.

16. A catheter comprising:
a shaft to be inserted into a blood vessel, the shaft being flexible and having a tubular shape, wherein:
the shaft comprises:
a light emitting portion that is located at a distal end portion of the shaft and is configured to emit near-infrared light by being irradiated with light, and
a non-light emitting portion that extends from the light emitting portion toward a proximal end side of the shaft and does not emit the near-infrared light; and
a length of the light emitting portion in an extending direction of the shaft is six times or less an outer diameter of the non-light emitting portion in an extending direction of the shaft.

17. The catheter according to claim 16, wherein:
the shaft comprises a shaft body;
the non-light emitting portion is formed by a portion of the shaft body; and
the light emitting portion comprises a light emitting layer on at least one of an inner surface or an outer surface of a distal end portion of the shaft body.

18. The catheter according to claim 16, wherein:
the shaft comprises a shaft body;
the non-light emitting portion is formed by a portion of the shaft body, and
the light emitting portion comprises a light emitting wall portion formed by inclusion of a light emitting material in a distal end portion of the shaft body.

19. A catheter assembly comprising:
the catheter according to claim 16; and
a needle body inserted into the catheter.

20. A catheter system comprising:
the catheter according to claim 16;
an irradiation unit configured to irradiate a living body site including a blood vessel into which the catheter is inserted with light;
a light receiving unit configured to receive the light guided from the living body site and the near-infrared light emitted by the light emitting portion; and
an image display unit configured to display a received light image created on a basis of the light and the near-infrared light received by the light receiving unit.
